(19) United States
(12) Patent Application Publication
(10) Pub. No.: US 2025/0281164 A1
(43) Pub. Date: Sep. 11, 2025

(54) SPECIMEN COLLECTION APPARATUS

(71) Applicant: Syramed Sciences, LLC, Liverpool, NY (US)

(72) Inventors: Robert Anthony Dracker, North Syracuse, NY (US); Robert A. Dracker, Liverpool, NY (US)

(21) Appl. No.: 19/071,512

(22) Filed: Mar. 5, 2025

Related U.S. Application Data
(60) Provisional application No. 63/561,585, filed on Mar. 5, 2024.

(51) Int. Cl. A61B 10/00; A61B 90/96; A61B 90/98

(52) U.S. Cl. CPC A61B 10/0096; A61B 90/96; A61B 90/98

(57) ABSTRACT

A specimen collection apparatus having an elongated tubular body, a first sealed end, a second open end and a flared member extending from the open end. The flared member acting to guide a specimen into the second open end and configured to fold over and adhere to the elongated tubular body thereby securing the specimen therein. The flared member additionally configured as a scoop or shovel to extract a specimen from an environment and place the specimen into the tubular body.

### CROSS-REFERENCE TO RELATED APPLICATION

The present application claims priority to and the benefit of U.S. Provisional Application No. 63/561,585, filed Mar. 5, 2024, the contents of which are incorporated herein by reference and made a part hereof.

### BACKGROUND OF THE INVENTION

The present invention generally relates to the collection of a specimen. More particularly the invention relates to a specimen collection apparatus configured to collect, retain, and identify a specimen.

Specimen are needed in variety of industries to collect a sample of an environment and to test for various environmental conditions. For medical use, a swab is used gather biological material to test for various viruses or infections. For military use, a swab may be used for the pathological or chemical testing of food preparation surfaces, battlefield forensics, criminal forensics, investigations, laboratory testing. For municipal use, a swab may be used for the pathological or chemical testing of food preparation surfaces, municipal water supply, municipal air exchange, internal and external forensics, criminal and crime scene forensics, investigations, or laboratory testing. In the context of industrial uses a swab may be used for the pathological or chemical testing of food preparation surfaces, compliance testing, formulaic testing, chemical composition testing, contaminant testing, or laboratory testing.

Various specimen collection apparatuses have been created over the years to keep the specimen from contamination and to keep the specimen secure. Often these solutions involve complex seals and custom molded plastic housings. Other variations comprise of wax/plastic lined paper subject to damage in travel or liquid permeations.

The present invention addresses the shortcomings of the prior art by presenting a specimen collection apparatus that is easy to handle, inexpensive to make, fluid impermeable, and easy to seal.

### SUMMARY OF THE INVENTION

In view of the above, the specimen collection apparatus comprises an elongated tubular body having a first end, a second end, and a flared member extending from the second end. The first end is sealed and is configured to retain a specimen. The second end comprises a specimen collection opening and is configured to receive the specimen. The flared member is configured to fold over the specimen collection opening to seal the specimen collection opening.

One of skill in the art would note that a specimen may be a swab as shown in the figures, but may also be any material from the environment including but not limited to soils, liquids, oils, biological materials, or industrial samples placed into the collection apparatus.

In one aspect, the flared member is configured as a scoop or shovel such that the flared member can be used collect the specimen itself into the tubular body.

In one aspect, the flared member further comprises an adhesive configured to adhere the flared member to the tubular body and at least partially seal the specimen collection opening securing the specimen inside.

In another aspect, the specimen collection apparatus further comprises an adhesive label configured to adhere the flared member to the tubular body and seal the specimen collection opening, securing the specimen inside. The adhesive label may be used alone to secure the flared member or in combination with an adhesive applied to the flared member as described above.

The invention further comprises a method of securing a swab specimen comprising the steps of inserting a specimen into the specimen collection opening of the specimen collection apparatus, allowing the specimen to bottom out on first sealed end of the specimen collection apparatus, exposing adhesive coupled to the specimen collection apparatus flared member, sealing the specimen collection opening by folding the flared member over the specimen collection opening and adhering the flared member to the specimen collection apparatus.

### BRIEF DESCRIPTION OF THE DRAWINGS

In the accompanying figures, like elements are identified by like reference numerals among the several preferred embodiments of the present invention.

FIG. 1 illustrates an example front view of a specimen collection apparatus.

FIG. 2 illustrates an example rear view of a specimen collection apparatus.

FIG. 3 illustrates an example of a specimen being inserted into a specimen collection apparatus.

FIG. 4 illustrates an example of a specimen being inserted into a specimen collection apparatus.

FIG. 5 illustrates an example of a specimen being inserted into a specimen collection apparatus.

FIG. 6 illustrates an example of a specimen loaded onto the flared member.

FIG. 7 illustrates an example of an adhesive label sealing a specimen collection apparatus.

FIG. 8 illustrates an example of an adhesive label sealing a specimen collection apparatus.

Other aspects and advantages of the present invention will become apparent upon consideration of the following detailed description, wherein similar structures have similar reference numerals.

### DETAILED DESCRIPTION OF THE PREFERRED EMBODIMENTS

The foregoing and other features and advantages of the invention will become more apparent from the following detailed description of an exemplary embodiment, read in conjunction with the accompanying drawings. The detailed description and drawings are merely illustrative of the invention rather than limiting, the scope of the invention being defined by the appended claims and equivalents thereof.

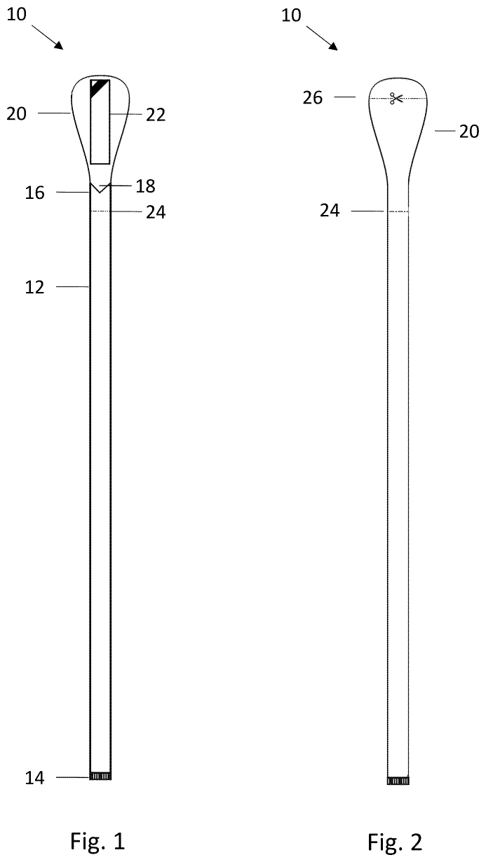

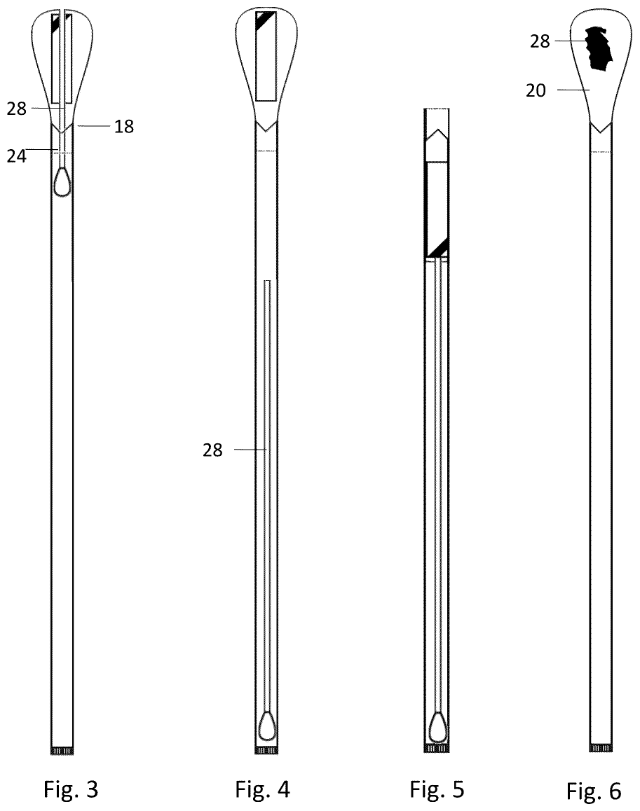

FIG. 1 and FIG. 2 show a front and rear view of one aspect of the specimen collection apparatus 10. As shown, the specimen collection apparatus 10 comprises a tubular body 12, having a first end 14, a second end 16, a specimen collection opening 18, and a flared member 20 extending from the second end 16. The first end 14 is sealed and is configured to act as a retaining wall for the specimen 28. As shown in FIG. 3 and FIG. 4, the specimen collection opening 18 is configured to receive the specimen 28.

As shown in FIG. 1, FIG. 2, and FIG. 5 the flared member 20 may further comprise an adhesive 22. In one aspect, the flared member 20 may fold over the specimen collection opening 18. In some instances a pre-fold indent 24 may be integrated into the flared member 20 or tubular body 12 to aid in the folding process. In operation as shown in FIG. 5, the flared member 20 is folded over and adhered through adhesive 22 to the tubular body 12 and at least partially seals the specimen collection opening 18 securing the specimen 28 inside. The flared member 20 may also act as a funnel or taper to ease the specimen 28 into the specimen collection opening 18.

In another aspect as shown in FIG. 6 the flared member 20 may be configured as a shovel or scoop to facilitate the transfer of specimen 28 into the tubular body 12. In some aspects, the flared member 20 may be more rigid than the tubular body 12 to facilitate the scooping or shoveling action. Further in some aspects, the flared member 20 may be rotatably coupled to the tubular body 12.

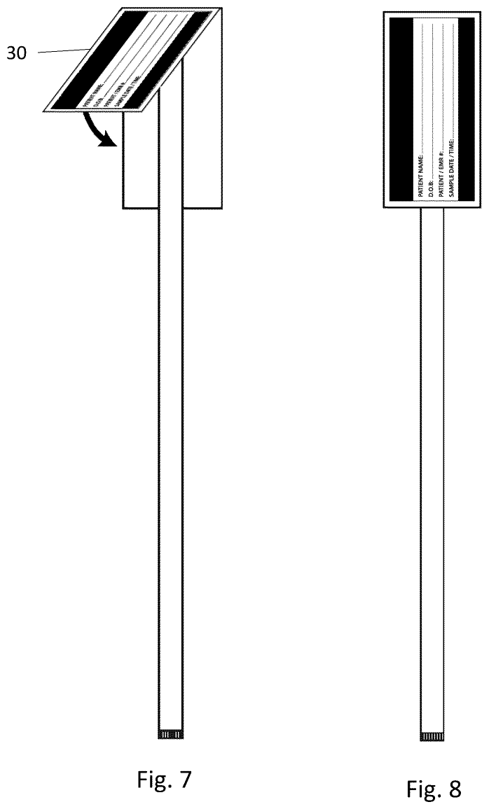

In another aspect, as shown in FIG. 7 and FIG. 8 the specimen collection apparatus 10 further comprises an adhesive label 30 configured to adhere the flared member 20 in a folded position to the tubular body 12 and further seal the specimen collection opening 18 securing the specimen 28 inside. The adhesive label 30 may comprise specimen identity indicia, an identifying barcode or QR code, or even an wireless transmitting device including but not limited to an RFID/BLE/NFC identifier tag to, track and geolocate the specimen 28 in the proper effort to maintain chain-of-custody of the specimen 28 as needed.

In yet another aspect, the tubular body 12 comprises a circular or oblong cross-section. The circular or oblong cross-section may be configured with a diameter or cross-section to accommodate a standard specimen swab 28.

In some aspects the apparatus 10 comprises a plastic including but not limited to HDPE, LDPE, polypropylene, polystyrene, polycarbonate, acrylic, or nylon. In other aspects the apparatus 10 may comprise a plastic, wax, or metallic film lined paper The tubular body 12 may be transparent, semi-transparent, or opaque. The material of the apparatus 10 may be fluid impermeable. In some aspects the material may also be treated with chemical agents or coatings to facilitate the sterilization or proper condition for storage or preparation of the enclosed specimen 28.

In yet another aspect, the flared member 20 or sealed end comprise a perforated portion or cut-line indicia 26 configured to be cut or torn away for removal of the specimen 28. The invention further comprises a method of securing a specimen comprising the

steps of inserting a specimen 28 into the specimen collection opening 18 of the specimen collection apparatus 10, allowing the specimen 28 to bottom out on first end 14 of the specimen collection apparatus 10, exposing adhesive 22 coupled to the flared member 20, sealing the specimen collection opening 18 by folding the flared member 20 over the specimen collection opening 18 and adhering the flared member 20 to the specimen collection apparatus 10. The method may further comprise the steps of adhering an identifying label 30 to the tubular body 12 and folding the label 30 over the flared member 20 further securing the flared member 20 and specimen 28 therein.

In another aspect, the invention further comprises a method of securing a specimen 28 comprising the steps of using the flared member 20 to scoop a specimen 28 from an environment into the specimen collection opening 18 of the specimen collection apparatus 10, allowing the specimen 28 to bottom out on first sealed end 14 of the specimen collection apparatus 10, exposing an adhesive 22 coupled to the specimen collection apparatus flared member 20, sealing the specimen collection opening 18 by folding the flared member 20 over the specimen collection opening 18 and adhering the flared member 20 to the specimen collection apparatus 10. The process may further include the step of sealing the flared member 20 with the adhesive label 30 in addition to or instead of the flared member adhesive 22.

Those of ordinary skill in the art will understand and appreciate the aforementioned description of the invention has been made with reference to a certain exemplary embodiment of the invention, which describe a specimen collection apparatus method of use. Those of skill in the art will understand that obvious variations in construction, material, dimensions or properties may be made without departing from the scope of the invention which is intended to be limited only by the claims appended hereto.

1. A specimen collection apparatus comprising:
a tubular body, having a first end and a second end;
a flared member extending from the second end;
the first end sealed;
the second end having a specimen collection opening; and
the flared member configured to fold over the specimen collection opening thereby at least partially sealing the tubular body.

2. The specimen collection apparatus of claim 1 wherein the first end is heat sealed together.

3. The specimen collection apparatus of claim 1 wherein the flared member further comprises an adhesive assembled to an inside surface of the flared member wherein the adhesive is configured to adhere to the tubular body upon the flared member being folded over.

4. The specimen collection apparatus of claim 1 further comprising an identifying adhesive label assembled to the tubular body.

5. The specimen collection apparatus of claim 4 wherein the identifying adhesive label is assembled to the second end and folded over the flared member thereby securing the flared member to the tubular body.

6. The specimen collection apparatus of claim 4 wherein the identifying adhesive label comprise specimen identifying indicia, a barcode, a QR code, or wireless transmitter.

7. The specimen collection apparatus of claim 1 wherein the tubular body comprises a plastic or paper lined with wax, plastic, or a metallic film.

8. The specimen collection apparatus of claim 1 wherein the tubular body is chemically treated or coated to facilitate sterilization of the tubular body or proper storage of the specimen.

9. The specimen collection apparatus of claim 1 wherein the specimen collection apparatus is fluid impermeable.

10. The specimen collection apparatus of claim 1 wherein the tubular body or the flared member further comprise a perforated portion or cut-line indicia configured to unseal the specimen collection apparatus.

11. The specimen collection apparatus of claim 1 wherein the tubular body comprises a pre-fold indentation configured to aid in the fold over of the flared member.

12. The specimen collection apparatus of claim 1 wherein the flared member is configured as a scoop or shovel shape configured to facilitate collection of an environmental specimen.

13. The specimen collection apparatus of claim 12 wherein the flared member is rotatably coupled to the tubular member.

14. The specimen collection apparatus of claim 12 wherein the flared member is more rigid than the tubular member.

15. The specimen collection apparatus of claim 12 wherein the flared member is more rigid than the tubular member.

16. A method of securing a specimen comprising the steps of:
obtaining a specimen collection apparatus comprising a tubular body, having a first end and a second end, a flared member extending from the second end, the first end sealed, the second end having a specimen collection opening, and the flared member configured to fold over the specimen collection opening thereby at least partially sealing the tubular body;
inserting a specimen into the specimen collection opening and into the tubular body; and
at least partially sealing the tubular body by folding over the flared member over the specimen collection opening.

17. The method of securing a specimen of claim 16 wherein the step of inserting the specimen further comprises using the flared member to scoop the specimen from an environment and facilitate the specimen entering the specimen collection opening.

18. The method of securing a specimen of claim 16 wherein the step of at least partially sealing the tubular body further comprises the step of adhering the flared member to the tubular body.

19. The method of securing a specimen of claim 16 wherein the step of at least partially sealing the tubular body further comprises the step of adhering an identifying adhesive label to the tubular body over the flared member.

20. The method of securing a specimen of claim 19 further including the step of tracking the specimen through the identifying adhesive label through identifying indicia, a barcode, a QR code, or wireless transmitter.
